(19) United States
(12) Patent Application Publication
(10) Pub. No.: US 2025/0017782 A1
(43) Pub. Date: Jan. 16, 2025

(54) METHOD FOR PROVIDING CONTROL DATA FOR AN OPHTHALMOLOGICAL LASER OF A TREATMENT APPARATUS FOR HYPEROPIA CORRECTION

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/771,685

(22) Filed: Jul. 12, 2024

(30) Foreign Application Priority Data
Jul. 13, 2023 (DE) 10 2023 118 630.2

(51) Int. Cl. A61F 9/008

(52) U.S. Cl. CPC A61F 9/0084; A61F 9/00802; A61F 2009/00872

(57) ABSTRACT

Method for providing control data for an ophthalmological laser (12) of a treatment apparatus (10) for hyperopia correction of a cornea (16). As steps, the method includes ascertaining (S10) corneal data of the cornea (16) from predetermined examination data; determining (S12) correction parameters of the hyperopia correction and corneal parameters of a virtual cornea, which is assumed for the cornea (16) after treatment with the hyperopia correction, depending on the ascertained corneal data; determining (S14), if a preset limiting criterion is present for at least one correction parameter of the hyperopia correction and/or at least one corneal parameter of the virtual cornea, wherein exceeding at least one parameter limit in the hyperopia correction is examined by the limiting criterion; limiting (S16) at least one preset correction parameter of the hyperopia correction by a limit value if the limiting criterion is present; and providing (S18) the control data, which includes the hyperopia correction with the limited correction parameter.

### FIELD

The invention relates to a method for providing control data for an ophthalmological laser of a treatment apparatus for hyperopia correction of a cornea. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea of an eye are known in the prior art. Therein, pulsed lasers and a beam focusing device may, for example, be formed such that laser pulses effect a photodisruption and/or ablation in a focus area situated within the organic tissue to remove a tissue, in particular a tissue lenticule, from the cornea.

In correcting hyperopia (long-sightedness), the range of possible parameters of the laser and/or parameters of the cornea may be restricted, which means that an originally planned or desired correction is not realizable or would result in disadvantageous effects for the eye, since it is outside of technical and/or physiological limits. These limits may often be unknown for users, wherefore treatments outside of possible or reasonable correction parameters are planned.

### SUMMARY

It is the object of the invention to facilitate planning for a hyperopia correction.

This object is solved by the independent claims. Advantageous embodiments of the invention are disclosed in the dependent claims, the following description as well as the figures.

The invention is based on the idea that correction parameters, which are outside of optimized limits for the treatment, are automatically adapted or limited to improve a treatment. Therein, it may in particular be provided that a first correction parameter, which is outside of the optimized limit, is not limited, but another parameter, which is dependent on the first correction parameter.

A first aspect of the invention relates to a method for providing control data for an ophthalmological laser of a treatment apparatus for hyperopia correction of a cornea, wherein the method comprises the following steps performed by a control device. Therein, an appliance and/or an appliance component, in particular a computer or processor, which may automatically or semi-automatically perform the following steps, is to be understood by a control device: ascertaining corneal data of the cornea from predetermined examination data; determining correction parameters of the hyperopia correction and corneal parameters of a virtual cornea, which is assumed for the cornea after treatment with the hyperopia correction, depending on the ascertained corneal data; determining if a preset limiting criterion is present for at least one correction parameter of the hyperopia correction and/or at least one corneal parameter of the virtual cornea, wherein exceeding at least one parameter limit in the hyperopia correction is examined by the limiting criterion; limiting at least one preset correction parameter of the hyperopia correction by a limit value if the limiting criterion is present; and providing the control data, which includes the hyperopia correction with the limited correction parameter.

In other words, corneal data may be determined from predetermined examination data such as for example topography measurements and/or wavefront measurements, which specify an actual state of the cornea before the treatment. From this corneal data, in which a hyperopia may in particular be present, correction parameters may then be determined, which are to be applied for treating the hyperopia. Furthermore, a virtual cornea may be determined, in which it is assumed that the cornea, which is characterized by the corneal data, has been treated by the correction parameters. This means, the virtual cornea represents an assumed postoperative cornea.

Subsequently, it may be determined, if a preset limiting criterion is present in the planned correction parameters and/or in the corneal parameters or the virtual cornea generated thereby. The limiting criterion may include one or more conditions or rules, wherein the control device may examine the respective correction parameters and/or corneal parameters to the effect whether or not they are respectively satisfied. Therein, the limiting criterion may be present if at least a parameter limit in the correction parameters and/or a parameter limit in the virtual cornea are exceeded. For example, the limiting criterion may be present for the correction parameters if contradictory inputs are performed in planning a volume body to be removed and/or a value has been selected too high in planning the hyperopia correction. The limiting criterion may be present in the virtual cornea if it is, for example, determined that in the virtual cornea, a residual thickness is too low in a position and/or a too high curvature is generated by the treatment such that the stability of the cornea after the treatment is for example compromised.

One or more parameters may be limited in response to a limiting criterion being met. For example, if a first correction parameter and/or corneal parameter is above a preset parameter limit, a second correction parameter may be restricted to a value or a range of values, which may be optimized for the treatment apparatus. Furthermore, in response to the limiting criterion being met, the preset parameter limit which was exceeded may be reset to a different limit value, such that that one or more parameters are then limited by the limit value. In other words, a correction parameter or corneal parameter may in some cases be allowed to exceed a preset parameter limit, and may be limited to a limit value which is different from the preset parameter limit. In addition to limiting the correction parameter, or, for example, if a parameter near the parameter limit is determined, a warning may also be output. This means that a warning may be generated for a user, which makes him aware of the parameter limit, in particular already just before the limiting criterion is present and/or if the preset correction parameter is limited. For example, the warning may be output via a user interface of the treatment apparatus. Then, a user may decide if the treatment is to be overall stopped or if it is continued with the limited correction parameter, or if the originally planned correction parameter, which is outside of the parameter limit, is to be maintained and thus the limitation is cancelled.

Finally, control data for controlling the ophthalmological laser may be provided, which may be generated based on the limited correction parameter for the hyperopia correction.

For example, the correction parameters may include values for a refractive power change or refraction change for hyperopia correction, a size and/or position of a treatment zone, in particular of an optical zone, a laser energy and/or a pulse distance. The corneal parameters of the virtual cornea may for example include a residual thickness of the cornea after treatment, achieved curvature values, aberration values and/or achieved refraction values.

By the invention, the advantage arises that planning a hyperopia correction is facilitated for a user since correction parameters may be adjusted or limited to preset limits in automated manner. This also increases a safety in the treatment since erroneous adjustments of parameters may thus be reduced.

The invention also includes embodiments, by which additional advantages arise.

In an embodiment, the limiting criterion is present if the virtual cornea is thinner in a periphery, or at a diameter, than in a center. Herein, the virtual cornea radially viewed is meant by center and periphery, wherein a center may be situated in the area of a pupil central point and/or a corneal vertex and the periphery radially spaced therefrom. In particular, in the hyperopia correction, there is the risk that a periphery is too severely thinned compared to the center, which may be avoided hereby. Therein, the entire periphery does not have to be thinner when azimuthally viewed, but the limiting criterion may be present if, for example, a preset portion of the periphery, for example, more than 10 percent of the periphery, is thinner than the center.

In particular, a warning is generated if the virtual cornea is thinner at the periphery, at any diameter, than 1.2 times a center thickness. This may be examined from a radial position starting from the center such that, for example, upon presence of one of the following pairs of values, which specify the diameter and the respective ratio of the thickness at the center to the thickness at the respective diameter, the limiting criterion is present: (6 mm; 1.15), (7 mm; 1.175), (8 mm; 1.2), (9 mm; 1.225) and (10 mm; 1.25).

In particular, a warning is generated if the virtual cornea is thinner at a diameter than a preset ratio compared to its center. For example, upon presence of one of the following pairs of values, which specify the diameter and the respective ratio of the thickness at the center to the thickness at the respective diameter, the limiting criterion is present: (6 mm; 1.15), (7 mm; 1.175), (8mm; 1.2), (9 mm; 1.225) and (10 mm; 1.25). In other words, a warning is generated if, at a diameter of 6 mm of the cornea, a portion of the cornea at that diameter is thinner than 1.15 times the thickness at the center of the cornea; a warning is generated if, at a diameter of 7 mm of the cornea, a portion of the cornea at that diameter is thinner than 1.175 times the thickness at the center of the cornea; a warning is generated if, at a diameter of 8 mm of the cornea, a portion of the cornea at that diameter is thinner than 1.2 times the thickness at the center of the cornea; a warning is generated if, at a diameter of 9 mm of the cornea, a portion of the cornea at that diameter is thinner than 1.225 times the thickness at the center of the cornea; and a warning is generated if, at a diameter of 10 mm of the cornea, a portion of the cornea at that diameter is thinner than 1.25 times the thickness at the center of the cornea. At any diameter, if a present portion of the periphery of the virtual corner is thinner than the center, the limiting criterion is present.

In a further embodiment, the limiting criterion is present if an average curvature of the virtual cornea exceeds 49 diopters. In other words, the limiting criterion is present if the average curvature of the virtual cornea, that is averaged over all meridians, as it is expected after the treatment, is greater than 49 diopters. This value was regarded as critical from follow-up examinations.

In particular, a warning is generated if an average curvature of the virtual cornea exceeds 47 diopters. This means that a warning is already generated above 47 diopters, before the preset correction parameter is limited by a limit value at 49 diopters.

In a further embodiment, the limiting criterion is present if a curvature of a meridian of the virtual cornea exceeds 50 diopters. This means that at least one correction parameter is limited if a single meridian of the virtual cornea has a curvature of above 50 diopters.

In particular, a warning is generated if a curvature of a meridian of the virtual cornea exceeds 48 diopters.

In a further embodiment, the limiting criterion is present if the ascertained spherical aberration of the virtual cornea falls below −0.7 diopters. In other words, the limiting criterion is present if the spherical aberration is less than −0.7 diopters, for example is at −0.8 diopters.

In particular, a warning is generated if an ascertained spherical aberration of the virtual cornea falls below −0.5 diopters.

In a further embodiment, a warning is generated if an average refraction change by the hyperopia correction exceeds 6 diopters. In other words, a warning message may be provided if it is provided as the correction parameter that a refraction change above 6 diopters is to be performed.

In a further embodiment, a warning is generated if a refraction change of a meridian of the virtual cornea exceeds 8 diopters.

In a further embodiment, if the limiting criterion is present, a diameter of a treatment zone, which is defined by an optical zone and a transition zone, is limited to a value as the correction parameter. The value is limited to less than a lamella diameter value, wherein the lamella diameter value is defined by a diameter of a planned foldable corneal lamella subtracted with a hinge height, which the corneal lamella has at a remaining joint with the cornea. In other words, upon presence of the limiting criterion, the diameter of the treatment zone may be limited such that it is smaller than the diameter of a foldable corneal lamella, which is additionally subtracted with the hinge height. Thus, the formula may for example be used:

OZ+TZ<lamella diameter value−hinge height,

wherein OZ is the optical zone and TZ is the transition zone. In addition, a constant value may also be subtracted from the lamella diameter value, which is in the range between 0 and 0.7 millimeters, in particular 0.5 millimeters, to consider a tolerance range.

In a further embodiment, if the limiting criterion is present, a diameter of a treatment zone, which is defined by an optical zone and a transition zone, is limited to a value as the correction parameter. The value is limited to smaller than a corneal diameter value wherein the corneal diameter value is defined by a diameter of the cornea subtracted with a double offset value of a pupil center to a corneal vertex. In other words, the value, to which the treatment zone is limited, may be the corneal diameter value, wherein it may be defined with the formula:

OZ+TZ<diameter of the cornea−2*offset of pupil center to the corneal vertex

wherein a constant value may additionally be subtracted, which is in the range from 0.75 millimeters to 1.25 millimeters, wherein the constant value may secure a distance to a limbus of the cornea.

In a further embodiment, a diameter of an optical zone is limited to a value as the correction parameter, which is above the limit value of 6 millimeters, if the limiting criterion is present. This means that the optical zone of a treatment zone may be set greater than or equal to 6 millimeters.

In a further embodiment, if the limiting criterion is present, a diameter of a treatment zone, which is defined by an optical zone and a transition zone, is limited to a value as the correction parameter. The value is above the limit value of 7 millimeters. In other words, the treatment zone may be limited to greater than or equal to 7 millimeters if the limiting criterion is present.

A further aspect relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The control data may include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser may be included in the control data.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect of the invention relates to a control device, which is formed to perform the steps of at least one embodiment of the previously described methods. Thereto, the control device may comprise a computing unit for electronic data processing such as, for example, a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect of the invention relates to a treatment apparatus with at least one eye surgical or ophthalmological laser and a control device, which is formed to perform the steps of at least one embodiment of a previously described method. The respective laser may be formed to at least partially separate a predefined corneal volume with predefined interfaces of a human or animal eye by optical breakdown, in particular at least partially separate it by photodisruption and/or to ablate corneal layers by (photo)ablation and/or to effect a laser-induced refractive index change in the cornea and/or the eye lens and/or to increase a crosslinking of the cornea.

In a further embodiment of the treatment apparatus according to the invention, the laser may be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown may be spatially narrowly limited such that a high incision accuracy is allowed in the generation of the interfaces. In particular, the range between 700 nm and 780 nm may also be selected as the wavelength range.

In a further embodiment of the treatment apparatus according to the invention, the control device may comprise at least one storage device for at least temporary storage of at least one control dataset, wherein the control dataset or datasets include(s) control data for positioning and/or for focusing individual laser pulses in the cornea; and may comprise at least one beam device for beam guidance and/or beam shaping and/or beam deflection and/or beam focusing of a laser beam of the laser.

A further aspect of the invention relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster may access the computer-readable medium and read out the content thereof. The storage medium is for example formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the embodiments of another one of the aspects of the invention. Thus, the features of the embodiments of the invention may be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, configurations are also to be regarded as encompassed and disclosed by the invention, which are not explicitly shown or explained in the figures, but arise from and may be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram according to an exemplary embodiment.

In the figures, identical or functionally identical elements are provided with the same reference characters.

### DETAILED DESCRIPTION

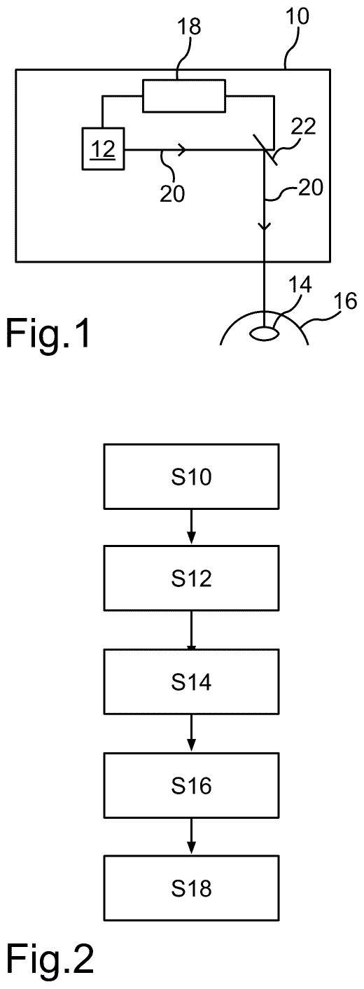

FIG. 1 shows a schematic representation of a treatment apparatus 10 with an ophthalmological laser 12 for removing a tissue 14 from a human or animal cornea 16 by photodisruption and/or ablation. For example, the tissue 14 may represent a lenticule or also volume body 14, which may be separated from the cornea 16 by the eye surgical laser 12 for correcting a visual disorder, in particular hyperopia. A correction profile or a geometry of the volume body 14 to be removed may be provided by a control device 18, in particular in the form of control data, which comprises correction parameters, such that the laser 12 emits pulsed laser pulses in a pattern predefined by the control data into the cornea 16 of the eye, to remove the volume body 14. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22 such as, for example, a rotation scanner, to remove the volume body 14. The beam deflection device 22 may also be controlled by the control device 18 to remove the volume body 14.

In particular, the illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example, between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

If the volume body 14 is removed from the cornea 16 within the scope of a hyperopia correction, some restrictions in the correction parameters for removing the volume 14 are to be considered. In particular, if certain correction parameters or corneal parameters to be expected of a postoperative cornea exceed limit values in the treatment planning, other correction parameters have to be adapted for compensation in order not to impair the treatment success. This means that correlations may be present between the individual correction parameters, which have to be considered, but which are not obvious in treatment planning. In order to provide these limitations in the hyperopia correction, the method shown in FIG. 2 may be performed.

FIG. 2 shows a schematic method diagram for providing control data for an ophthalmological laser 12 of a treatment apparatus 10 for hyperopia correction of a cornea 16. For example, the method may be performed by the control device 18 of the treatment apparatus 10 or by an external control device 18.

In a step S10, corneal data of the cornea 16 may be ascertained from predetermined examination data thereto. Herein, the corneal data represents an actual state of the cornea, which may have hyperopia.

In a step S12, a correction of the hyperopia may be planned depending on the ascertained corneal data, wherein correction parameters, in particular a refraction change, may be determined hereto. Furthermore, it may be estimated in a step S12, how the cornea 16 will look after the treatment with the correction parameter, such that a virtual post treatment cornea with respective corneal parameters may be determined hereto.

In a step S14, it may then be determined if at least one correction parameter of the planned hyperopia correction and/or at least one corneal parameter, which the virtual cornea has after the hyperopia correction, is above a limit value or a parameter limit, wherein the presence of at least one limiting criterion for the correction parameters and/or the corneal parameters may be examined.

With respect to the corneal parameters of the virtual cornea to be expected, it may, for example, be examined if in the virtual cornea, a thickness in a periphery of the cornea is thinner in than in a center. Herein, it may, for example, be examined if, viewed in radial direction from the center at a radius of 3 millimeters, the periphery is 1.15 times thinner than the center, 1.175 times at 3.5 millimeters, 1.2 times at 4 millimeters, 1.225 times at 4.5 millimeters and 1.25 times at 5 millimeters. Furthermore, it may be examined if an average curvature of the virtual cornea exceeds 49 diopters, wherein, if the average curvature exceeds these 49 diopters, the limiting criterion may be present. Furthermore, the limiting criterion may be present if a curvature of a single meridian of the virtual cornea exceeds 50 diopters. The limiting criterion may also be present if a spherical aberration of the virtual cornea falls below −0.7 diopters, thus for example has −0.8 diopters.

In a step S16, one or more parameters may be limited in response to a limiting criterion being met. Furthermore, in response to the limiting criterion being met, the preset parameter limit which was exceeded may be reset to a different, limit value, such that that one or more parameters are then limited by the limit value. In other words, a correction parameter or corneal parameter may in some cases be allowed to exceed a preset parameter limit, and may be limited to a limit value which is different from the preset parameter limit.

In particular, if the limiting criterion is present, a diameter of a planned treatment zone, which is defined by an optical zone and a transition zone, may be limited as the correction parameter such that the limit value is above 7 millimeters. Alternatively or additionally, it may be provided that the optical zone is limited to above 6 millimeters if the limiting criterion is present.

Alternatively or additionally, the treatment zones may be limited to a corneal diameter value as the correction parameter, which is defined by the diameter of the cornea subtracted with a double offset value of a pupil center to a corneal vertex; or the treatment zone may be limited to a value below a lamella diameter value, which is defined by a diameter of a planned foldable corneal lamella subtracted with a hinge height, which the corneal lamella has at the remaining joint with the cornea.

In addition, a warning message may be output upon approaching a parameter limit, which makes a user aware of the approaching limitation. Thus, the user may decide if he modifies the correction parameter, stops the treatment planning, or further uses the correction parameter, which is above the parameter limit. A warning message may in particular be output if the average curvature of the virtual cornea exceeds 47 diopters, if a curvature of a single meridian of the virtual cornea exceeds 48 diopters, a spherical aberration of the virtual cornea falls below −0.5 diopters, an average refraction change by the hyperopia correction exceeds 6 diopters and/or if a refraction change of a single meridian of the virtual cornea exceeds 8 diopters.

In contrast, if a limiting criterion is not present, the initially determined correction parameters may be used for hyperopia correction.

However, if the limiting criterion is present and one of the mentioned correction parameters has been limited, the hyperopia correction with the limited correction parameter may be provided by the control device 18 in a step S18, in that control data is generated, which may control the laser 12 and/or the beam deflection device 22 for performing the hyperopia correction. This means that the treatment apparatus 10 may be controlled by the control data.

Overall, the examples show how parameter limits of the hyperopia correction may be taken into account by the invention.

1. A method for providing control data for an ophthalmological laser of a treatment apparatus for hyperopia correction of a cornea, wherein the method comprises the following steps performed by a control device:
ascertaining corneal data of the cornea from predetermined examination data;
determining correction parameters of the hyperopia correction and corneal parameters of a virtual cornea of the hyperopia correction, depending on the ascertained corneal data;
determining if a limiting criterion is present for at least one correction parameter of the hyperopia correction and/or at least one corneal parameter of the virtual cornea, wherein a limiting criterion is present if the at least one correction parameter or the at least one corneal parameter exceed a parameter limit;
in response to meeting the limiting criterion, limiting at least one correction parameter of the hyperopia correction by a limit value; and
providing the control data, which includes the hyperopia correction with the limited correction parameter.

2. The method according to claim 1, wherein the limiting criterion is present if the virtual cornea is thinner in a periphery than in a center.

3. The method according to claim 1, wherein a warning is generated if the virtual cornea is thinner in a periphery than 1.2 times a center.

4. The method according to claim 1, wherein the limiting criterion is present if an average curvature of the virtual cornea exceeds 49 diopters.

5. The method according to claim 1, wherein a warning is generated if an average curvature of the virtual cornea exceeds 47 diopters.

6. The method according to claim 1, wherein the limiting criterion is present if a curvature of a meridian of the virtual cornea exceeds 50 diopters.

7. The method according to claim 1, wherein a warning is generated if a curvature of a meridian of the virtual cornea exceeds 48 diopters.

8. The method according to claim 1, wherein the limiting criterion is present if an ascertained spherical aberration of the virtual cornea falls below −0.7 diopters.

9. The method according to claim 1, wherein a warning is generated if an ascertained spherical aberration of the virtual cornea falls below −0.5 diopters.

10. The method according to claim 1, wherein a warning is generated if an average refraction change by the hyperopia correction exceeds 6 diopters.

11. The method according to claim 1, wherein a warning is generated if a refraction change of a meridian of the virtual cornea exceeds 8 diopters.

12. The method according to claim 1, wherein if the limiting criterion is present, a diameter of a treatment zone, as one of the at least one correction parameters, which is defined by an optical zone and a transition zone, is limited to a value which is smaller than a lamella diameter value, wherein the lamella diameter value is defined by a diameter of a planned foldable corneal lamella subtracted with a hinge height, which the corneal lamella has at a remaining joint with the cornea.

13. The method according to claim 1, wherein, if the limiting criterion is present, a diameter of a treatment zone, as one of the at least one correction parameters, which is defined by an optical zone and a transition zone, is limited to a value which is smaller than a corneal diameter value, wherein the corneal diameter value is defined by a diameter of the cornea subtracted with a double offset value of a pupil center to a corneal vertex.

14. The method according to claim 1, wherein a diameter of an optical zone is limited to a value as the correction parameter, which is above the limit value of 6 mm, if the limiting criterion is present.

15. The method according to claim 1, wherein a diameter of a treatment zone, which is defined by an optical zone and a transition zone, is limited to a value as the correction parameter, which is above the limit value of 7 mm, if the limiting criterion is present.

16. A control device, which is configured to perform a method according to claim 1.

17. A treatment apparatus with at least one eye surgical laser for the separation of a corneal volume with predefined interfaces of a human or animal eye by optical breakdown, in particular by photodisruption and/or photoablation, and at least one control device according to claim 16.

18. (canceled)

19. A computer-readable medium for storing a computer program, the computer program comprising commands which cause a treatment apparatus to execute a method according to claim 1.
